(19) United States
(12) Patent Application Publication
(10) Pub. No.: US 2019/0077663 A1
(43) Pub. Date: Mar. 14, 2019

(54) BLACK PHOSPHOROUS AND PHOSPHORENE-BASED MOLECULAR ANALYSIS DEVICES AND RELATED METHODS

(71) Applicants: THE TRUSTEES OF THE UNIVERSITY OF PENNSYLVANIA, Philadelphia, PA (US); Rensselaer Polytechnic Institute, Troy, NY (US)

(72) Inventors: Marija DRNDIC, Jr., Philadelphia, PA (US); Paul C. MIASIH DAS, Philadelphia, PA (US); Vincent MEUNIER, Cohoes, NY (US)

(21) Appl. No.: 16/085,201

(22) PCT Filed: Mar. 15, 2017
(86) PCT No.: PCT/US2017/022411

§ 371 Date: Sep. 14, 2018

Related U.S. Application Data
(60) Provisional application No. 62/308,897, filed on Mar. 16, 2016.

(51) Int. Cl. C01B 25/02; G01N 33/487

(52) U.S. Cl. CPC C01B 25/02; B01L 2200/12; G01N 33/48721

(57) ABSTRACT

The present disclosure provides black phosphorous devices with use in, e.g., inter alia, molecular analysis applications. A device may comprise a region of black phosphorous with, e.g., 1 to 10 layers and having a pore formed through the layer or layers. The black phosphorous may be supported by a support membrane. Also provided are related methods of molecular analysis and methods of fabricating the devices.

### RELATED APPLICATION

This application claims priority to and the benefit of U.S. patent application no. 62/308,897, “Black Phosphorous-Based Molecular Analysis Devices and Related Methods” (filed Mar. 16, 2016), the entirety of which application is incorporated herein by reference for any and all purposes.

### GOVERNMENT RIGHTS

This invention was made with government support under R21HG007856 awarded by the National Institutes of Health and under EFRI-1542707 awarded by the National Science Foundation. The government has certain rights in this invention.

TECHNICAL FIELD

The present disclosure relates to the field of molecular measurement and to the field of black phosphorous devices.

### BACKGROUND

With advances in molecular detection and medical diagnostics has also come a need for a platform technology having a flexible design that can be adapted to a variety of molecular analysis applications. There is also a related need for methods of using and fabricating such improved devices.

### SUMMARY

Black phosphorus (known as BP and also known as phosphorene-phosphorene may be viewed as a single layer of black phosphorus) is a highly anisotropic allotrope of phosphorus with application to functional electronics and optoelectronics. Demonstrated here is that high-resolution and controlled structural modification of few-layer BP along arbitrary crystal direction can be achieved with nanometer-scale precision on a few-minute timescales leading to the formation of sub-nm wide armchair and zigzag BP nanoribbons. Nanoribbons may be assembled, along with nanopores and nanogaps, in a variety of ways, e.g., by using a combination of mechanical-liquid exfoliation and in situ transmission electron microscope (TEM) and scanning TEM nanosculpting.

Provided here are some exemplary time-dependent structural properties of the one-dimensional systems under electron irradiation and their oxidation properties with electron energy-loss spectroscopy (EELS). Also demonstrated here is the use of STEM to controllably narrow and thin the nanoribbons until they form into nanogaps, e.g., by breaking. The observations are rationalized using density functional theory for transition state calculations and electronic band-structure evolution for the various stages of the narrowing procedure.

Without being bound to any particular theory, sub- and few-nm wide BP nanoribbons realized experimentally possess clear one-dimensional quantum confinement, even when the systems are made up of a few layers.

As described herein, nanopores, nanoribbons, and nanogaps in suspended few-layer black phosphorus (BP) flakes may be formed using in situ transmission electron microscope nanosculpting. Few-layer BP flakes may be produced through a liquid exfoliation procedure and suspended on holey SiNₓ membranes. As shown herein, high-resolution structural modification of nanopores and nanoribbons can be achieved with nanometer-scale precision on timescales of a few minutes. Density functional theory may be used to provide a model for the observed anisotropy in edge formation by computing energy barriers for various edge geometries.

In meeting the long-felt needs in the art, the present disclosure first provides analysis devices, comprising: a portion of black phosphorous (BP) having a region that comprises one or more (e.g., from 1 to about 100) layers of black phosphorous, the region having at least one pore formed therehrough, and the at least one pore having a cross-sectional dimension in the range of from about 1 to about 100 nm.

Also provided are methods of molecular analysis, comprising: contacting an analysis device according to the present disclosure to a sample comprising one or more molecules; translocating one or more of the molecules through the pore of the analysis device; and monitoring a signal related to the translocation of the molecule through the pore.

Additionally provided are methods of fabricating an analysis device, comprising: disposing an amount of black phosphorous on a support membrane, the amount of black phosphorous having one or more (e.g., from 1 to about 100) layers; and forming a pore in a region of the black phosphorous.

Further provided are methods of molecular analysis, comprising: contacting an analysis device comprising a pore formed in a region of black phosphorous to a sample comprising one or more molecules; translocating one or more of the molecules through the pore of the analysis device; and monitoring a signal related to the translocation of the molecule through the pore.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 provides an exemplary characterization of few-layer BP flakes and the elliptical expansion of nanopores: (a) optical micrograph of a holey SiNₓ membrane used to perform AFM measurements after few-layer BP deposition. (b) AFM image of a suspended few-layer sample and an AFM linescan across the SiN_{X}/BP interface indicating a 13-nm-thick flake. The inset is an HRTEM image (200 kV) of the BP flake indicated by the white square in (a). Inset scale bar is 1μμm (1 micrometer). The white region in (b) corresponds to a thick, folded region of the BP flake and appears more opaque in the TEM, as expected. (c) STEM simulation (top) and illustration (bottom) of AB-stacked bilayer BP with zigzag and armchair lattice constants a₁ and a₂, respectively, in addition to half-lattice-constants a₁′ and a₂′. A and B layers are highlighted in different colors. (d) HAADF STEM image of the few-layer BP lattice indicating spacings that are consistent with a₁′ (200) and a₂′ (020) (the inset is the corresponding FFT). (e) HRTEM image and corresponding SAED pattern (inset) of a few-layer BP flake suspended on a holey carbon grid. (0 HRTEM image of the region indicated by the white square in (e), containing a 10-nm-diameter nanopore with dimensions L₂₀₀ and L₀₂₀ in the zigzag and armchair directions, respectively. Over a period of 15 minutes of electron irradiation (current density=6.1×10⁻² pA/nm²), the pore expanded elliptically with eccentricity evolving from (f) L₂₀₀:L₀₂₀=1.0 to (g) 1.2, (h) 1.3, and (i) 1.4. All scale bars in (f) to (i) are 5 nm.

FIG. 2 provides exemplary structure and energetics of the phosphorene edges as computed using density functional theory. (a) Overhead views of the armchair (AC), zigzag single-termination (ZZ-1), zigzag double-termination (ZZ-2), inner-shifted reconstruction of the ZZ-2 edge (ZZRC-i), and outer-shifted reconstruction of the ZZ-2 edge (ZZRC-o) phosphorene edges. The corresponding edge energies are given in eV/Å to quantify the thermodynamic stability. (b) Energy landscapes for removing a single atom from phosphorene edges via the nudged elastic band method. (c) Schematic illustrating the observation of an elliptical nanopore in phosphorene. The armchair edges (black) recede faster than the most stable zigzag edge (ZZRC-o) due to the greater energy barrier for removing atoms from the latter edge, which results in relatively longer nanopore dimensions in the zigzag (200) direction (FIG. 1f-i).

FIG. 3 illustrates STEM nanosculpting of zigzag and armchair few-layer BPNRs. (a)-(g) HAADF STEM (200 kV) images of an (a)-(g) 8.0-nm-long zigzag and (i)-(p) 6.5-nm-long armchair nanoribbon. (a),(i) In BPNRs of total width wᵣ, both crystalline regions with width w_{c} and amorphous edge regions with width wᵣ, were observed.

Thinning of the ribbons from (a) to (h) and from (i) to (p) was observed by using the HAADF intensity-thickness correlation for each panel along the red line X (indicated in (a) and (i), see also FIG. 5a). The ribbons were fabricated and narrowed by forming two nanopores and then nanosculpting the region between them. The insets in (b) and (j) are FFT's taken along the nanoribbon axes and indicate lattice spacings of 1.65 and 2.28 Å, which are consistent with a₁′ and a₂′, repsectively. For the zigzag case, wᵣ is initially (a) 7.2 nm and subsequently decreased to (b) 6.0, (c) 5.7, (d) 4.6, (e) 2.8, and (f) 2.2 nm. The corresponding w_{c} values are (a) 5.6, (b) 4.6, (c) 4.3, (d) 3.1, (e) 1.9, (f) 1.0, and (g) 0 nm. For the armchair case, the values for wᵣ are (a) 6.3, (b) 5.8, (c) 4.2, (d) 3.1, (e) 2.8, and (0 2.5 nm. The corresponding wₑ values are (a) 4.9, (b) 4.2, (c) 3.0, (d) 1.4, (e) 0.9, (f ) 0.5, and (g) 0 nm. After the BPNRs break, (h) 3.5- and (p) 2.8-nm-wide nanogaps remain. All scale bars in (a) to (p) are 5 nm.

FIG. 4 provides exemplary DFT-calculated electronic band structures with the Fermi level set to 0 eV of BPNRs based on the experimentally realized crystalline widths. The edge bands are indicated by EB with the number of such bands in parenthesis. The schematic diagrams on the left indicate the corresponding BPNR structure. (a) Single layer armchair. (b) Bulk armchair. (c) Single layer ZZ-1. (d) Bulk ZZ-1.

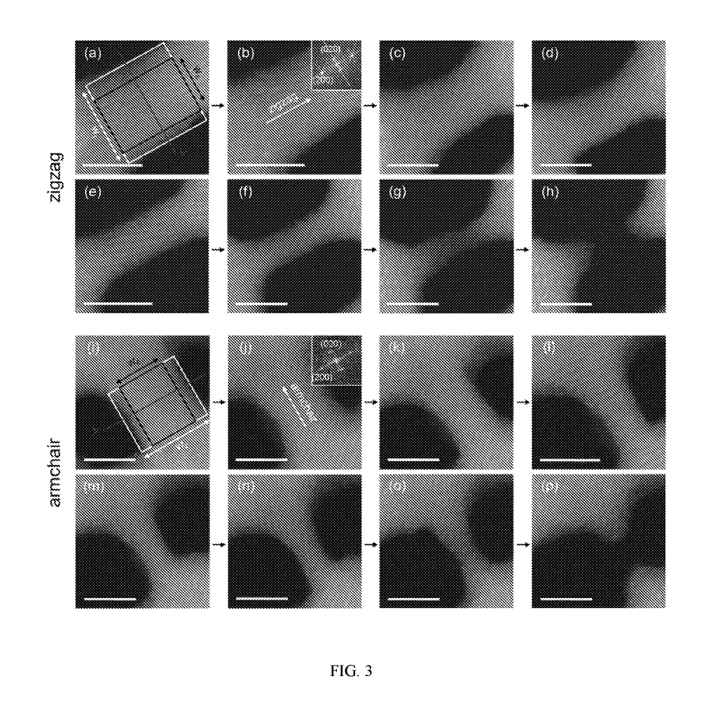

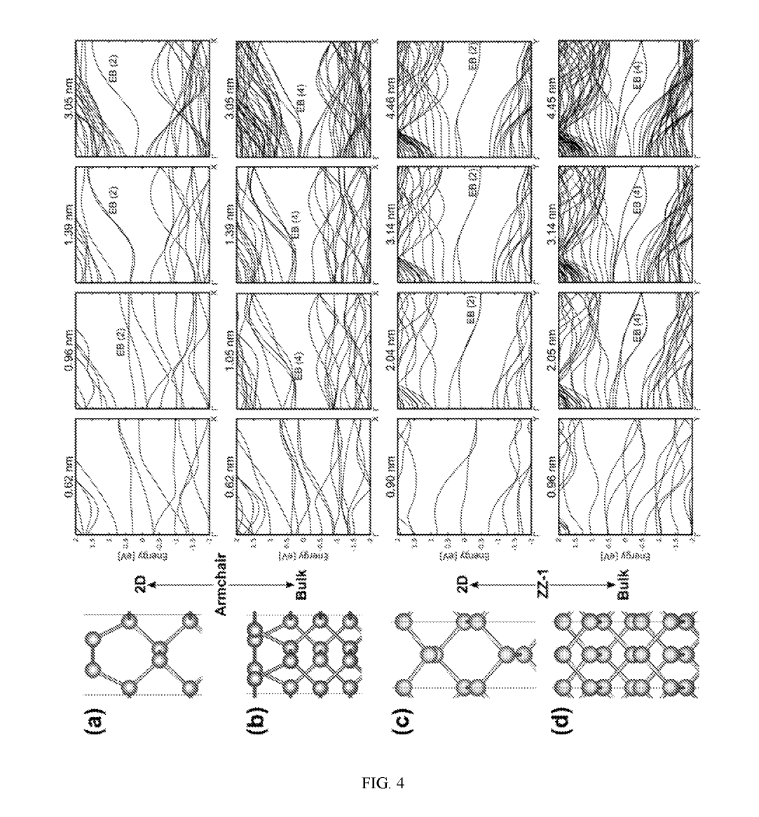

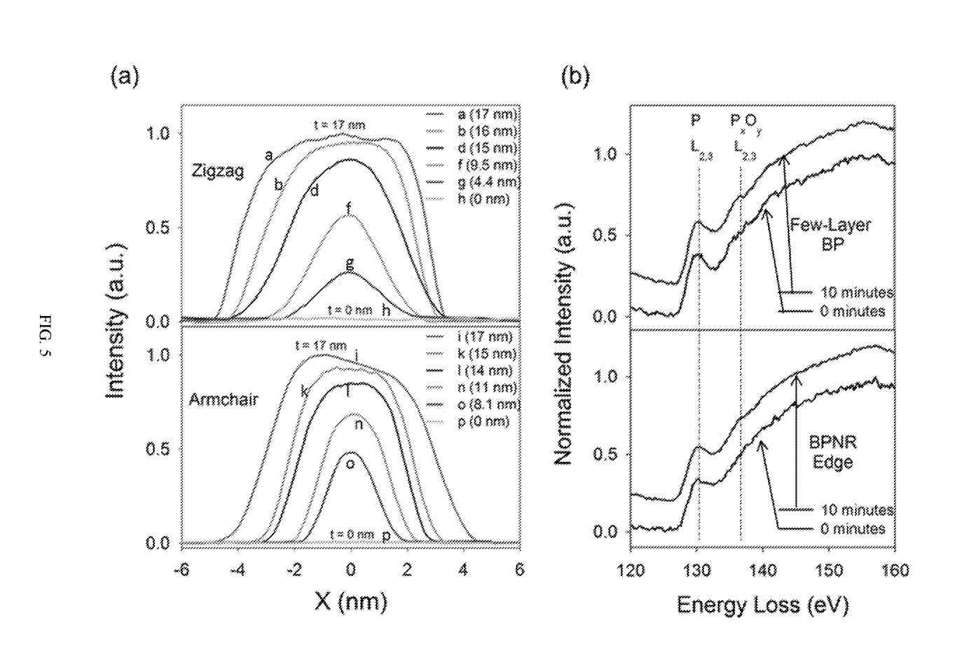

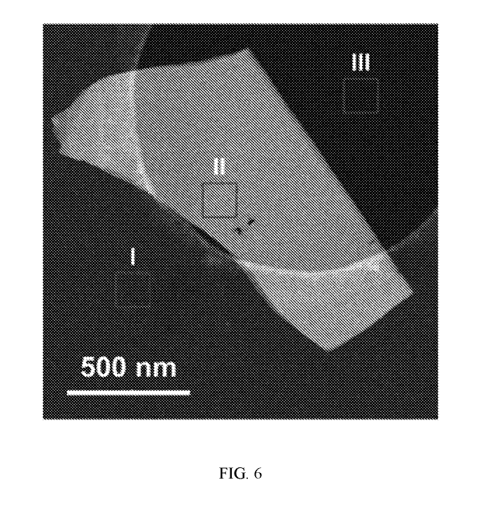

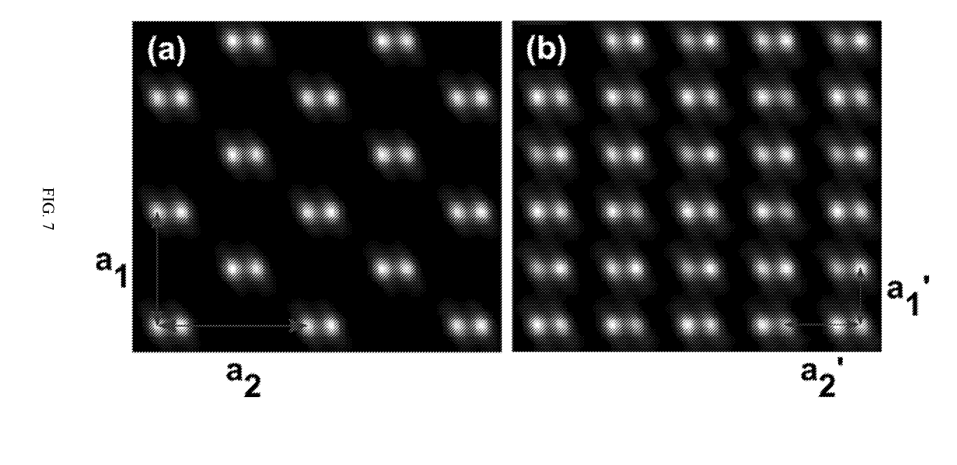

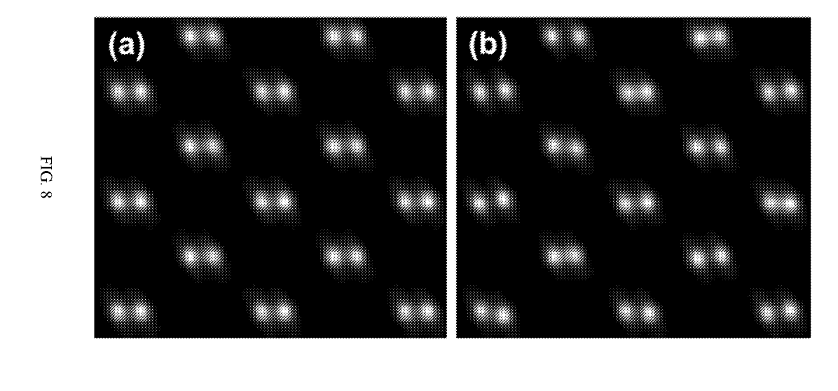

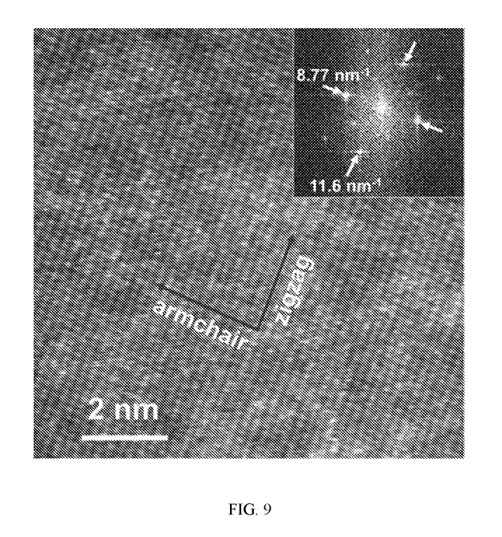

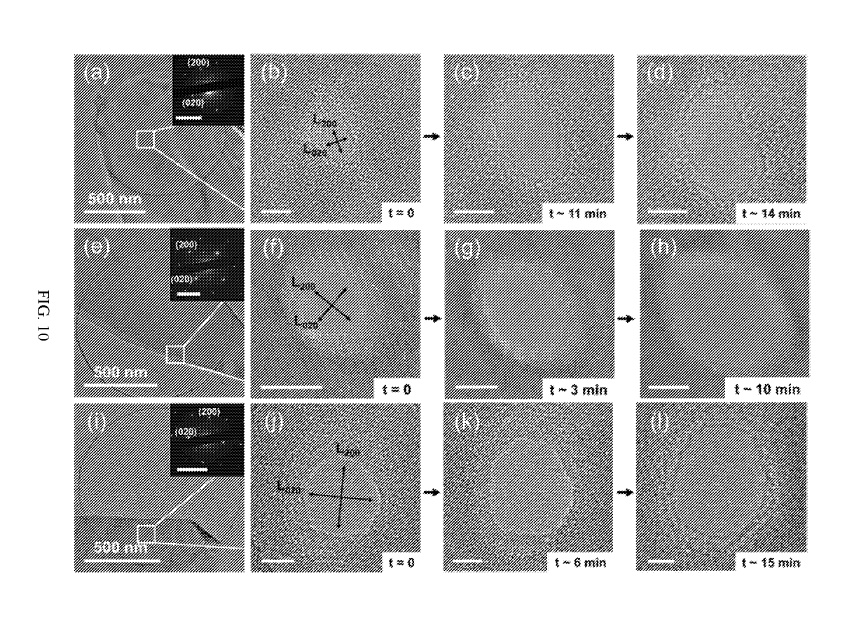

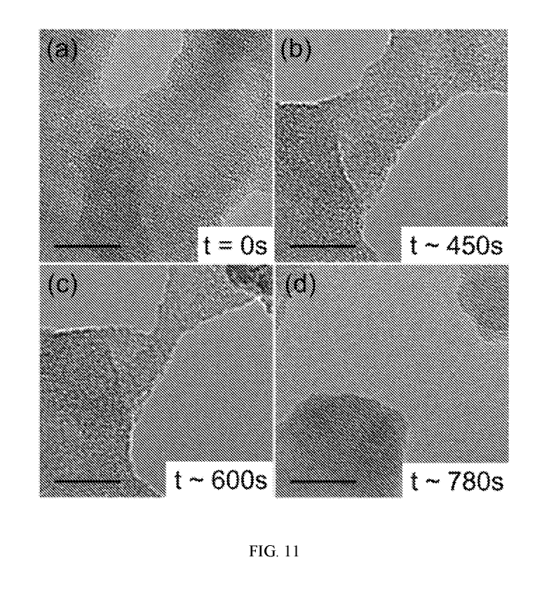

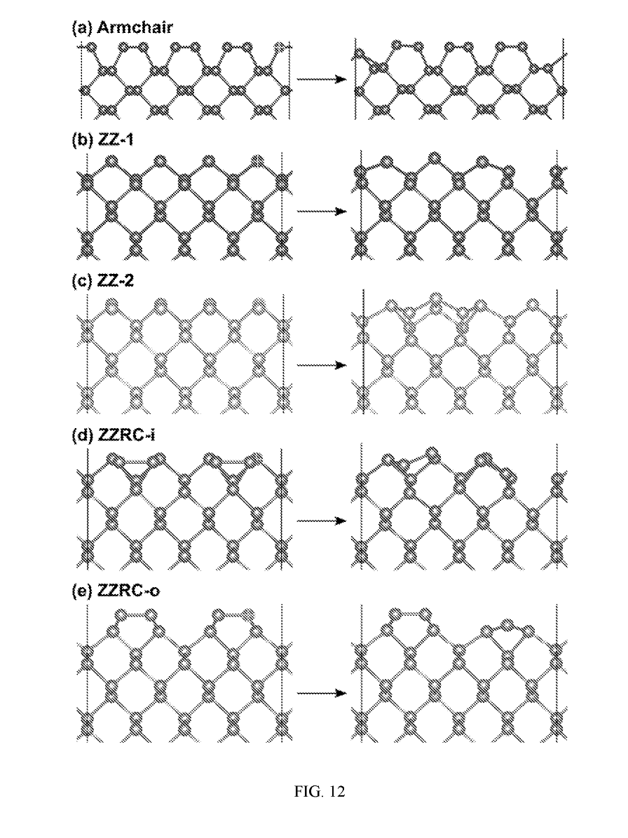

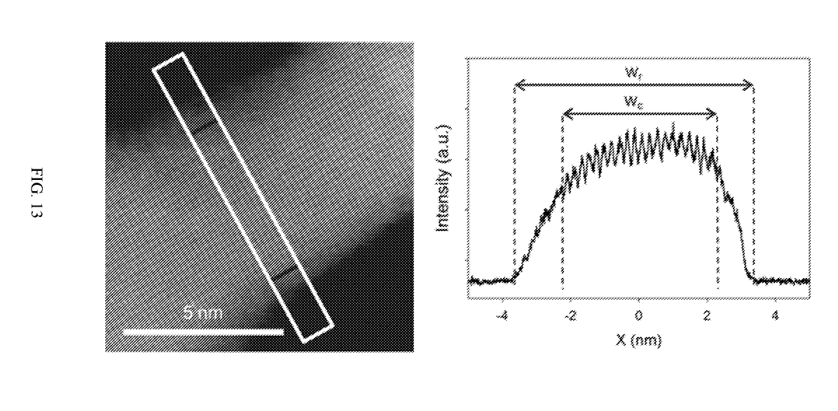

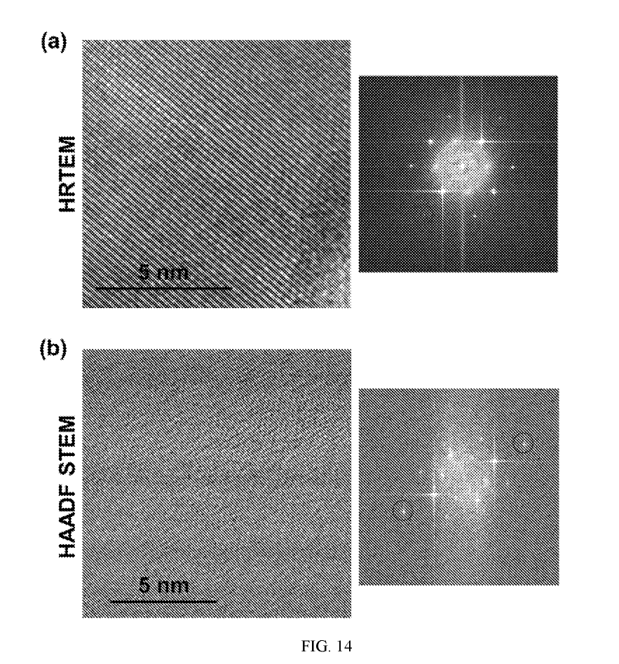

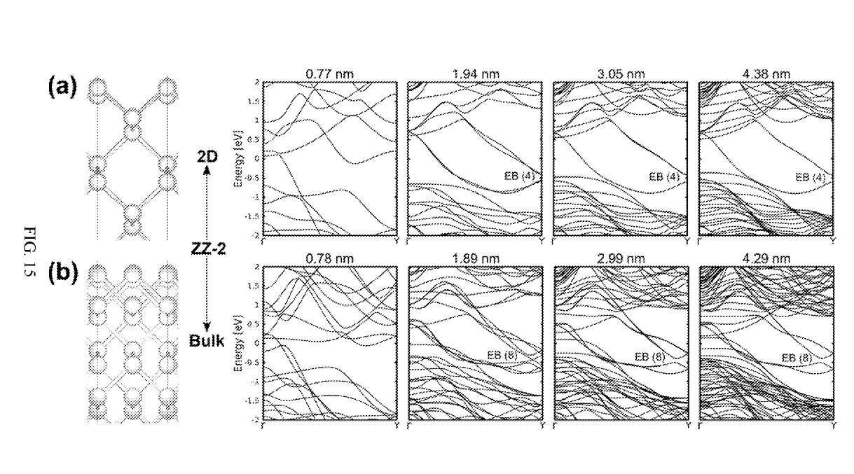

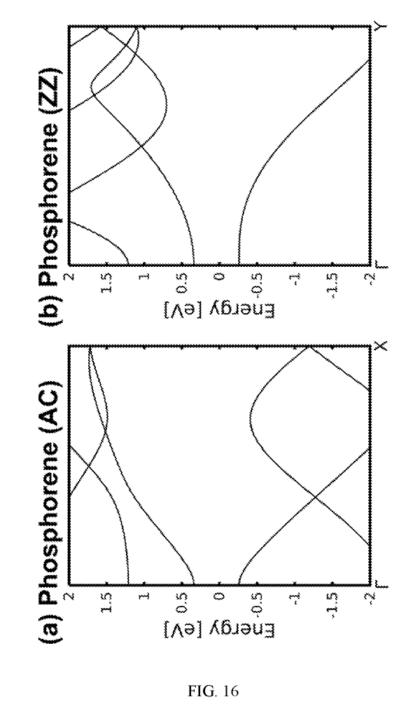

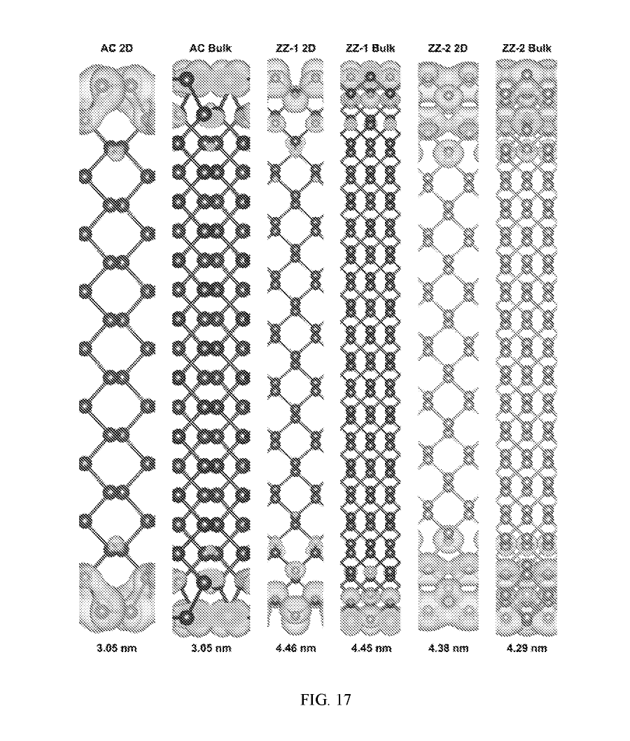

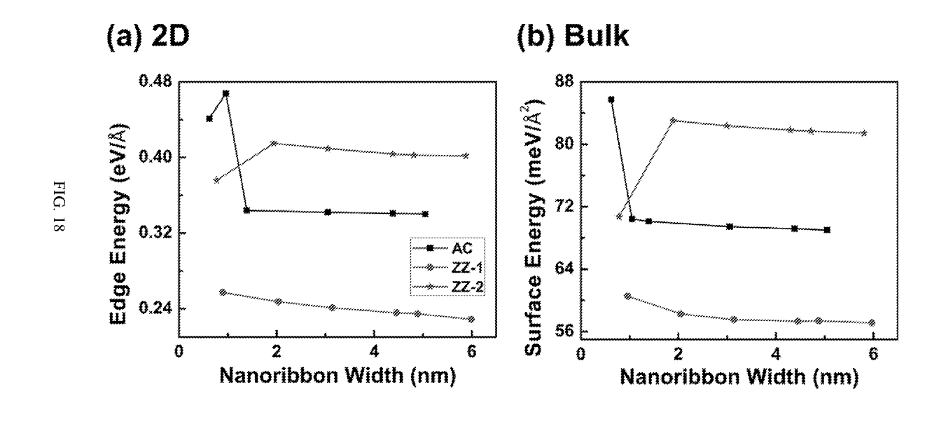

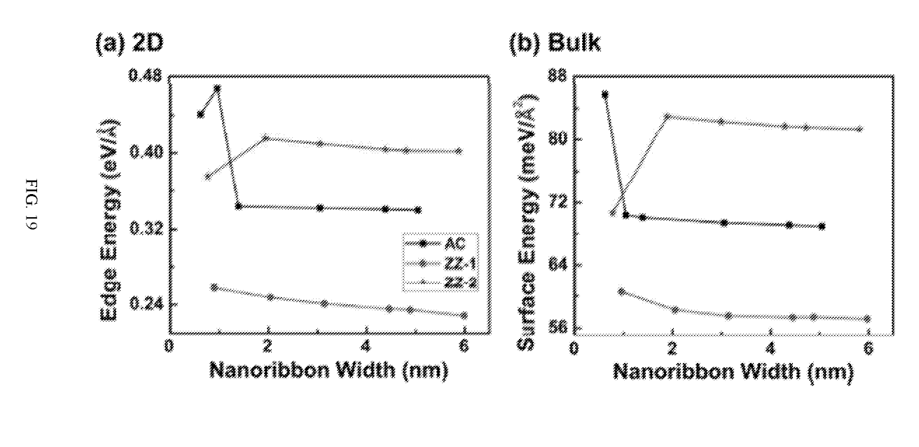

FIG. 5 provides exemplary thinning and EELS analysis of few-layer BPNRs. (a) The intensity cross-sections of the zigzag (top) and armchair (bottom) BPNR STEM micrographs that appear in FIG. 3 (red lines X as indicated in FIGS. 3a,i). Each linescan corresponds to a particular panel, where BPNR thickness t is determined through the HAADF intensity-thickness correlation discussed in the main text. From an initial thickness of 17 nm (Section S1), BPNRs were thinned down to thicknesses of 4.4 (zigzag) and 8.1 nm (armchair). (b) Core-loss EELS spectra for a 14-nm-thick few-layer BP flake (top) and the edge of a nanosculpted BPNR (bottom) are shown in blue. The corresponding spectra after 10 minutes of atmospheric oxidation are shown in red, showing the appearance of PₓO_{y}. The L_{2,3} edge for P and PₓO_{y} are indicated at 130.2 and 136 eV, respectively.

FIG. 6 provides a low-magnification HAADF STEM image of the 17-nm-thick BP flake used to fabricate the nanostructures in FIG. 3. Electron image intensities of the carbon film at I, few-layer BP flake at II, and background at III were used to ascertain the flake thickness using the simplified electron cross-section formula.

FIG. 7 provides STEM simulations of (a) phosphorene indicating full lattice constants (a₁,a₂) (b) AB stacked bilayer BP from single bulk BP unit cell indicating half lattice constants (a₁′,a₂′). The zigzag direction is along a₁ and a₁′ and the armchair direction is along a₂ and a₂′.

FIG. 8 provides STEM simulations of phosphorene (to determine the effect of thermal diffuse scattering (TDS)), (a) neglecting TDS and (b) including TDS at 293 K were generated. Both images include 100×100 pixels. The overall difference is negligible for determining structure and therefore TDS was neglected in generating the images in FIG. 7. This significantly reduces the computational effort since finalized images including TDS should be averaged over many runs.

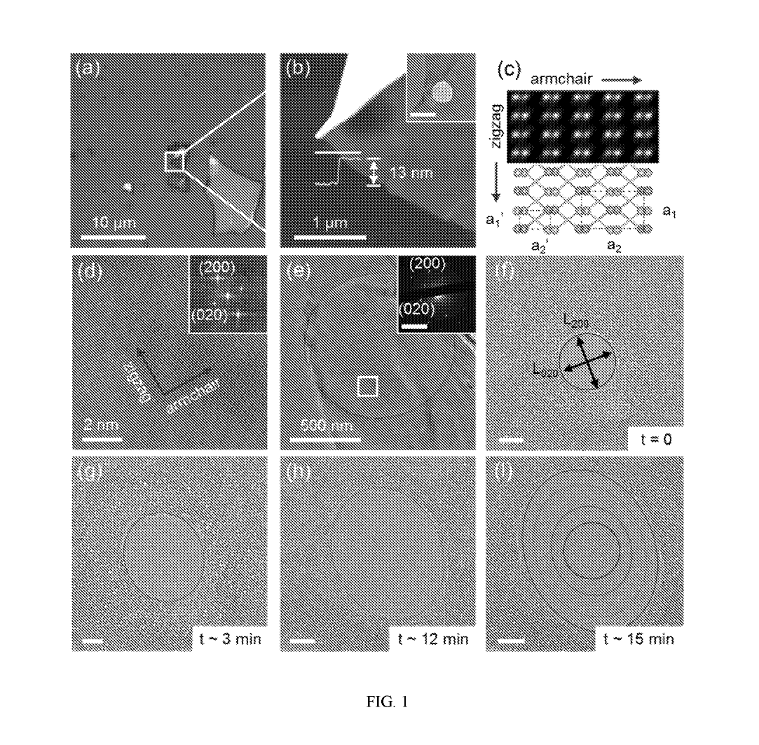

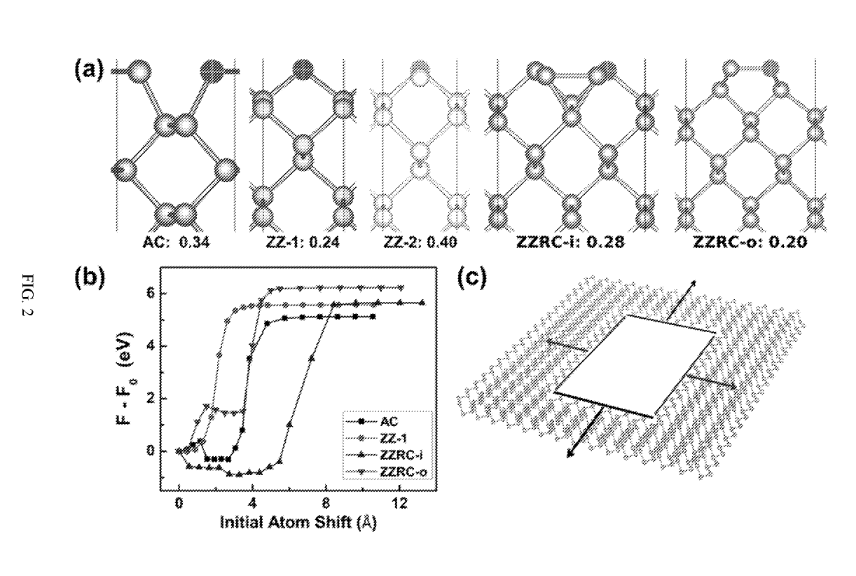

FIG. 9 provides 80 kV HAADF STEM few-layer BP lattice image and (inset) resulting FFT. As expected for the AB-stacked few-layer BP structure (FIG. 1c), the reciprocal lattice spacings of 11.6 and 8.77 nm⁻¹ correspond to a₁′ (1.67 Å) and a₂′ (2.24 Å), the real-space zigzag and armchair half-lattice-constants, respectively. This is in agreement with the results seen at 200 kV (FIG. 1d). The region of interest was tilted to the [001] zone axis prior to imaging.

FIG. 10 provides an exemplary elliptical expansion of suspended BP nanopores under constant electron beam irradiation under HRTEM (accelerating voltage=200 kV, current density=6.1×10⁻² pA/nm²) over a period of 10-15 minutes. Ratio between nanopore dimension along zigzag and armchair directions, i.e. L₂₀₀:L₀₂₀, evolved from 1.0 (FIG. 10b,f,j) to (c) 1.9 and (d) 2.0 for row 1, (g) 1.4 and (h) 1.5 for row 2, and (k) 1.2 and (1) 1.4 for row 3. Insets show corresponding diffraction patterns used to determine crystal orientation. All scale bars are 5 nm.

FIG. 11 provides an exemplary formation of a suspended few-layer BP nanoconstriction under electron irradiation over a period of 13 minutes. (a) HRTEM images of two nanopores separated by a distance of ˜20 nm. Constant irradiation (current density=6.1×10⁻² pA/nm²) narrows the area between the pores to form nanoconstrictions of narrowest width (b) 10 nm and (c) 3.8 nm until (d) the structure ultimately breaks off and yields a 15 nm gap. All scale bars are 10 nm.

FIG. 12 provides exemplary edge geometries of 4×1 supercells before and after removing single atoms (removed atom indicated by yellow crosshair).

FIG. 13 provides an exemplary intensity cross-section of the wᵣ (wₑ)=6.0 (4.6) nm zigzag BPNR from FIG. 3b. Cross-sections were used to measure wᵣ (total BPNR width) and w_{c} (crystalline region width). The region w_{c} is characterized by clear periodic fluctuations in intensity, which are indicative of a periodic atomic structure. The amorphous edge region region wᵣ-w_{c} also has non-zero intensity, but no periodic structure.

FIG. 14 provides Resolution limits for (a) TEM and (b) STEM imaging conditions were obtained by analyzing FFTs of the few-layer BP lattice. The resolution limit is obtained by analyzing the farthest discernible bright spots (red circles), the so-called transformation limit, in the resulting FFTs. For HRTEM (JEOL 2010F microscope) and HAADF STEM (JEOL ARM 200CF microscope), resolution limits of 0.11 and 0.08 nm, respectively, are reported.

FIG. 15 provides exemplary band structures with the Fermi level set to 0 eV for ZZ-2 edge black phosphorus nanoribbons based on the experimentally realized crystalline widths. The edge bands are indicated by EB with the number of such bands in parenthesis for (a) single layer and (b) bulk. All edge bands cross the Fermi level and therefore all nanoribbons are metallic.

FIG. 16 provides exemplary band structures for phosphorene along (a) armchair and (b) zigzag directions. The edge bands identified in the nanoribbon band structures are absent.

FIG. 17 provides exemplary band-specific charge densities for the edge bands corresponding to the largest width nanoribbons shown in FIG. 4 for armchair and ZZ-1, and FIG. 15 for ZZ-2.

FIG. 18 provides exemplary thermodynamic stability for armchair, ZZ-1, and ZZ-2 nanoribbons quantified by (a) the edge energy for single layer and (b) the surface energy for bulk.

FIG. 19 provides exemplary thermodynamic stability of 2D and bulk armchair, ZZ-1, and ZZ-2 nanoribbons as a function of width.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it can be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

Abbreviations: BP, black phosphorus; DFT, Density Functional Theory; and NEB, nudged elastic band.

Few-layer black phosphorus (BP) has attracted much attention over the past few years as an attractive novel two-dimensional (2D) nanomaterial owing to the compromise it offers between high carrier mobility and semiconducting properties found in other layered materials such as graphene and MoS₂, respectively. Specifically, BP features p-type semiconducting properties and reported carrier mobility values up to 1,000 cm² V⁻¹ s⁻¹. It is a mono-elemental realization of phosphorus with a structure characterized by stacked phosphorene monolayers held together by van der Waals forces. BP's basal plane consists of a highly anisotropic atomic structure of puckered atoms, as a consequence of the particular bonding allowed by phosphorus' 3s² 3p³ electronic shell structure which hybridizes into sp³ binding, three being singly occupied (and forming three bonds in-plane) and one doubly occupied.

This anisotropy results from the existence of two major symmetric directions with zigzag and armchair arrangements, respectively. The armchair (or “light”) direction features a high mobility of around 1,000 cm² V⁻¹ s⁻¹, while the zigzag (or “heavy”) direction has a much lower mobility of 600 cm² V⁻¹ s⁻¹. In the current state-of-the-art, bulk BP can be exfoliated down to a few layers via mechanical- or liquid-based methods. In comparison to other semiconducting few-atom-thick materials, such as 2D transition metal dichalcogenides (TMDs), few-layer BP devices exhibit in-plane anisotropic transport and a thickness-dependent direct bandgap (0.3-1.5 eV). These properties make few-layer BP an ideal candidate for optoelectronic applications.

Confining 2D materials into their nanoribbon counterparts is a suitable basis for developing 1D nanoelectronics. The fundamental principles of quantum mechanics suggest that properties of truly 1D assemblies depend critically on the details of the structure, and in particular on the associated confining potential, i.e. the materials' edge structure. Density functional theory (DFT) indicates that the transport properties of few-layer BP nanoribbons (BPNRs), such as bandgap magnitude and charge carrier's effective mass, are very sensitive to both ribbon width and crystallographic orientation due to BP's strongly anisotropic properties.

No study has been reported for the less stable BP materials in the few-layer regime. Unlike bottom-up approaches for other materials that have been pursued on the basis of the vast fundamental knowledge of organic chemistry, no such atom-by-atom approach exists or is likely to be developed soon for phosphorene due to its more complex chemistry which makes the structure vulnerable to environment. A number of phenomena such as the semiconductor-to-metal bandgap transition and Stark effect have been predicted for BPNRs but have yet to be experimentally investigated. Understanding these interesting phenomena first and foremost requires the ability to fabricate nanostructures with atomic control and with sizes small enough to enable the manifestation of quantum confinement.

Provided here is a top-down fabrication method that enables the first realization of nanoribbons, nanogaps, nanoconstrictions, and nanopores in few-layer BP. Using a TEM, one may drill nanopores in suspended few-layer BP flakes produced using a combined mechanical-liquid exfoliation procedure. Under uniform electron irradiation in TEM, measurements clearly show the orientation-dependent elliptical expansion of the pores at a controllable rate with nm-level precision into nanoconstrictions and nanogaps. Starting from the nanopores, one may fabricate few-nm wide armchair and zigzag BPNRs with sub-nm-precise STEM nanosculpting. Also investigated here are BPNR oxidation properties with electron energy-loss spectroscopy (EELS) and demonstrate the use of STEM to controllably narrow and thin the nanoribbons until they break into nanogaps. Further provided is DFT-based transition state modeling of various phosphorene edges and predict how the electronic properties of 1D BPNRs are governed by strong confinement effects.

Discussion

BP flakes were mechanically exfoliated from bulk BP crystals onto a SiO₂ substrate using blue Nitto tape. The flakes were further exfoliated by sonicating this SiO₂ substrate in dimethylformamide (DMF) for 60 minutes. Unexfoliated material remained on the tape, in contrast to the reported liquid exfoliation procedures which require centrifugation to remove bulk BP. The BP-DMF dispersions contain few-layer (<20 nm) flakes and are suitable for deposition onto TEM-compatible membranes, which cannot withstand harsh mechanical exfoliation techniques.

After sonication, BP-DMF dispersions were drop-cast onto holey carbon grids or holey silicon nitride (SiNₓ) membranes, dried in N₂, and annealed at 250° C. in Ar:H₂. Because of the detrimental effects on few-layer BP hole mobility, on/off ratio, and surface roughness from water and light-induced oxidation,²¹ all samples were stored in the dark, in vacuum (˜10 mTorr).

FIGS. 1a-d show the deposition of few-layer BP flakes and their structural characterization. Suspended few-layer BP flakes were identified using optical microscopy (FIG. 1a). Atomic force microscopy (AFM) scans of suspended flakes on holey SiNₓ membranes indicated sample thicknesses from 11 to 20 nm, including the 13 nm sample in FIG. 1b. Using high-angle annular dark-field (HAADF) STEM imaging, which produces mass-contrast images, flakes suspended on holey carbon TEM grids were found to have thicknesses in agreement with AFM results (FIG. 6). In addition to AFM and optical evidence, confirmed flake suspension was confirmed with TEM (FIG. 1b inset).

In few-layer BP, layers are stacked in an AB fashion (similar to bulk), in which adjacent layers are shifted by one half-lattice-constant in the zigzag direction. FIG. 1c includes a STEM simulation (top) and a schematic illustration (bottom) of this arrangement in bilayer BP. The STEM measurements report an image corresponding to projected positions of atoms of a few-layer deep. Due to BP's AB-stacked configuration, one observes structure with half-lattice-constants a₁′ (1.67 Å) and a₂′ (2.24 Å) compared to the BP basal plane lattice parameters a₁ (3.34 Å) and a₂ (4.47 Å) along the zigzag and armchair directions, respectively (FIG. 1d, Section S3). ^{24,25}

In the TEM, images are formed by irradiating a region of interest with an electron beam and monitoring transmitted electrons. The electron beam can also be focused into a small probe and used to drill nanopores. Here is shown for the first time that nanopores can be drilled in few-layer BP, as shown in FIG. le using high-resolution TEM (HRTEM). The selected area electron diffraction (SAED) pattern shown in the inset indicates the orthogonal orientation of the zigzag (200) and armchair (020) axes.

Nanopores such as the 10-nm-diameter one in FIG. if were drilled by focusing a TEM beam (probe size ˜1.0 nm, probe current=8.0 nA) for ˜1-2 seconds. Using the orientation of the SAED pattern, which agrees with orientations seen in high-resolution lattice images, L₂₀₀ and L₀₂₀ correspond to nanopore dimensions along the zigzag and armchair directions, respectively.

After initial drilling, the ratio of L₂₀₀:L₀₂₀ was 1.0 as expected for an approximately circular pore. Under an additional 15 minutes of electron beam irradiation (current density=6.1×10⁻² pA/nm²) in TEM imaging mode, L₂₀₀:L₀₂₀ is found to increase to a value of 1.4, showing preferential expansion in the zigzag direction (FIGS. 1f-i), leading to the expansion of the initially circular into an elliptical pore (see also FIG. 10). Moving beyond pore formation, the creation of nanoconstrictions and nanogaps in TEM was also observed by first drilling two adjacent pores and then simultaneously exposing them to broad electron beam irradiation in order to narrow the region between them. The time evolution for a nanoconstriction is provided in FIG. 11.

To explain the asymmetric opening of nanopores under symmetric electron beam irradiation, one may calculate the energy barriers for removing single atoms from several known phosphorene edges. One may employ first principles calculations within DFT and energy barrier evaluation using the nudged elastic band (NEB) method, as described elsewhere herein. Single unit cells of the five edges used and the corresponding edge energies of the supercells used are given in FIG. 2a. The edge energies were calculated as (F₀-NFₚ)/2L where F₀ is the total free energy of the system, N is the number of phosphorus atoms, Fₚ is the free energy per atom for phosphorene, and L is the lattice constant in the direction along the edge. Initial and final geometries for removing single atoms from various edges are given in FIG. 12, and animations for four specific cases are included in the SI. For this, three zigzag edges (ZZ-1, ZZRC-i, and ZZRC-o) and one armchair edge were considered. The resulting energy landscapes shown in FIG. 2b indicate that the overall energy barrier is the absolute value of the formation energy change itself. For armchair, ZZ-1, ZZRC-i, and ZZRC-o the corresponding energy barriers are 5.13, 5.57, 5.65, and 6.23 eV. Therefore, among these most stable structures, the ZZRC-o edge is the most resistant to the removal of a single atom.

Without being bound to any particular theory, for the ZZRC-i case the energy initially decreases because geometries resembling the more stable ZZRC-o edge are intermediate states. Again without being bound to any particular theory, for armchair the energy initially increases and then decreases below the zero reference level which suggests a reconstruction of the edge that is more stable than the standard armchair morphology. Even when factoring this additional decrease in energy into account, the energy barrier for the ZZRC-o edge is greater than that for the armchair reconstruction. Because the energy barrier for removing an atom from the most stable zigzag edge (ZZRC-o) is greater than that for the armchair edge, one may expect the armchair edge to recede faster under symmetric electron beam irradiation, leaving the zigzag edge relatively longer (L₂₀₀:L₀₂₀>1). This accounts for the development of the pores into elliptical shapes, as observed experimentally and illustrated schematically in FIG. 2c.

Here is provided a fabrication procedure for BPNRs in the few-layer thickness, sub-10-nm width regime based on STEM nanosculpting. The focused STEM beam can be rastered over a region of interest to form HAADF images or controllably maneuvered with sub-nm precision, where the electron beam can create clean cuts through a thin material (depending on the controllable electron energy).

The following strategy was used to fabricate few-nm wide BPNRs: first, two adjacent nanopores were nanosculpted in a 17-nm-thick BP flake (Section S1) with a focused STEM beam (probe size=0.15 nm, probe current=300 pA) and, second, the material was sculpted along either the zigzag or armchair direction until the material between them constituted a nanoribbon. In between periods of nanosculpting, the BPNRs were imaged by rastering the electron beam. While the BPNRs experienced thinning during STEM imaging, they remained crystalline unlike nanostructures exposed to orders of magnitude higher doses in TEM mode (see FIG. 11). In addition to crystalline regions of width w, amorphous edges with a roughly constant width wᵣ-w_{c} were also seen (FIGS. 3a,i), where wᵣ is the total width (See FIG. 13 for a definition of these quantities). This suggests localized few-layer BP lattice damage and/or small contamination during room-temperature nanosculpting, consistent with results for graphene. Nanosculpting works well for cutting through the entire stack (˜17 nm) of phosphorene layers, indicating that a fairly large amount of material is removed. However, lattice resolution is still achievable in STEM mode, showing the high quality of the nanoribbons. Measurements of wᵣ and w_{c} in addition to instrument resolution limits are discussed in Section S6. Also investigated was the time evolution of BPNRs, including their narrowing, thinning, and oxidation properties.

FIGS. 3a-h show the STEM nanosculpting of an 8.0-nm-long zigzag few-layer BP nanoribbon. From initial widths wᵣ(w_{c})=7.2 (5.6) nm in FIG. 3a, the ribbon was sculpted down to wᵣ(w_{c})=2.2 (1.0) nm (FIG. 30. The inset in FIG. 3b is a fast Fourier transform (FFT) of the crystalline region outlined in black in FIG. 3a and indicates a lattice spacing of 1.65 Å along the nanoribbon axis, which agrees well with the zigzag (200) half-lattice-constant a₁′ (1.67 Å). The width of the ribbon's amorphous edges (wᵣ-w_{c}) remained between 0.9 and 1.6 nm (FIGS. 3a-f). In FIG. 3f (wᵣ(w_{c})=2.2(1.0)nm), the structure was no longer nanosculpted but rather continuously imaged in STEM mode. After 160 seconds, the zigzag BPNR was narrowed to a 1.7-nm-wide amorphous (w_{c}=0 nm) structure and after an additional 40 seconds, snapped to form a 3.5-nm-wide nanogap (FIGS. 3g-h).

FIGS. 3i-p show the formation of a 6.5-nm-long armchair few-layer BP nanoribbon using a procedure similar to the one developed for the zigzag systems outlined above. From initial widths wᵣ(w_{c})=6.3(4.9) nm in FIG. 3i, the ribbon was sculpted down to wᵣ(w_{c})=2.5(0.5) nm (FIG. 3n). Similar to the zigzag case, the width of the ribbon's amorphous edges (wᵣ-w_{c}) remained fairly constant (between 1.2 and 2.0 nm). As indicated in the FFT (FIG. 3j, inset), the nanoribbon's axially-oriented lattice spacing of 2.28 Å is consistent with the armchair (020) lattice parameter a₂′ (2.24 Å). The wᵣ=2.1 nm amorphous (wᵣ=0 nm) ribbon in FIG. 3o was fabricated by allowing the wᵣ(w_{c})=2.5 (0.5) nm armchair BPNR in FIG. 3n to sit in STEM imaging mode (HAADF mode) for 80 seconds, similarly suggesting the possibility of narrowing sculpted BPNRs with sub-nm precision. A 2.8-nm-wide nanogap was formed after an additional 120 seconds of exposure (FIG. 3p).

To determine how narrowing induces quantum confinement in the range of widths achieved here, DFT-based band structures were calculated for single- and multi-layer armchair and zigzag-edged structures. For the latter were included different edge morphologies (ZZ-1, and ZZ-2). Details of the calculations can be found in elsewhere herein, and band structures for phosphorene along the same high symmetry directions are reproduced in FIG. 16. Only the four smallest non-zero crystalline widths obtained in the experiment for each edge are shown, since the overall structure of the bands changes negligibly (FIG. 4). The ZZ-1 and ZZ-2 edges were chosen for zigzag since the crystalline parts of the ribbons are surrounded by amorphous material, which in a first approximation would on average constrain the crystalline edge to the edges that would be formed from directly cutting the sheet or bulk. It is shown that in both 2D (i.e. single-layer) and bulk materials, the electronic band structure displays the 1D confinement effects as indicated by the presence of edge bands, which one may determine by calculating and plotting the partial charge density of bands near the Fermi level (FIG. 17). This finding results from the weak coupling between the individual phosphorene layers. A particularly interesting case is the ZZ-2 bulk 2.99 nm and 4.29 nm cases where at the Y point of the Brillouin zone two four-fold degeneracies exist. The edge energies (2D) and surface energies (bulk) are correlated with the widths in Section S10. Further modeling of the nanosculpting effect would require a systematic study of electron beam induced disorder in BP in addition to recent analysis of thermally induced defects.

In addition to nanosculpting as a method of narrowing BPNRs into the quantum confinement regime, ribbons were also found to be thinned during STEM imaging. To quantify both zigzag and armchair BPNR thicknesses, STEM intensity cross-sections were first obtained for each ribbon, as demonstrated in FIGS. 3a,i. Average fits were obtained for each BPNR width and peak values were correlated to BPNR thicknesses using a linear mono-elemental HAADF intensity-thickness relation I α, t, where I is the intensity cross-section peak value and t is the corresponding ribbon thickness. The resulting fits and corresponding thicknesses for both zigzag (FIGS. 3a-h) and armchair (FIGS. 3i-p) BPNRs are given in FIG. 5a. From an initial t=17 nm, the zigzag BPNR was reduced to a minimum of t=4.4 nm (8 layers) before breaking into a nanogap. Similarly, the armchair BPNR was reduced to a minimum thickness of 8.1 nm (14 layers). Thus, one may perform a thinning process and manipulation of BP's thickness by calibrating the dose of the electron exposure for a given BP stack thickness and width.

Because open edges are created during the narrowing procedure, one may consider this effect here as well. To determine the BPNR oxidation properties, reported here are EELS spectra for the edge of a nanosculpted BPNR (bottom) and the pristine parent few-layer flake (top) in FIG. 5b before and after 10 minutes at room temperature. Before oxidation, both the BPNR edge and few-layer BP flake show peaks at the L_{2,3} edge (130.2 eV) for phosphorus. The appearance of a peak at 136.0 eV, which has been suggested to be the PₓO_{y} L_{2,3} edge, is seen in the few-layer BP flake, and to a lesser extent in the BPNR edge, after the samples are removed from the TEM for 10 minutes and allowed to oxidize before reinsertion. This suggests that the edges of nanosculpted BPNRs exhibit different oxidative characteristics than the surfaces of few-layer BP flakes.

Demonstrated here is the first realization of 1D nanostructures in suspended few-layer BPNRs. Using TEM, one may nanosculpt nm-scale pores and showed their elliptical expansion under electron irradiation due to edge-dependent energy barriers for atomic removal. Also shown here is the first demonstration of sub-10-nm-wide zigzag and armchair BPNRs in the few-layer regime. Finally, using STEM-based nanosculpting and imaging, it was shown that BPNRs can be narrowed and thinned with sub-nm precision. Nanosculpting provides atomic resolution, sub-nm-precise fabrication possibilities, and is suitable for making nanostructures for fundamental studies and in situ characterization. The BPNR length can be extended similar to larger-scale TEM cutting of metals and graphene. STEM-based nanosculpting may also be useful for fabrication of multi-terminal devices with nm-scale features.

Methods

Few-layer BP exfoliation. Square arrays of 300-400 nm diameter holes spaced 10 μm apart were patterned in silicon nitride (SiNₓ) membranes with a focused ion beam (FIB). Few-layer BP dispersions were produced by mechanically exfoliating BP onto a SiO₂ substrate followed by sonication (Branson 2510, 40 kHz, 80 W) in dimethylformamide (DMF) for 1 hour at room temperature. The BP-DMF dispersions were drop-cast onto holey carbon TEM grids or SiNₓ membranes and then dried in 100% Ar for 12 hours at room temperature. Holey SiNₓ samples were used for AFM measurements while carbon grid samples were used in TEM, STEM, and EELS. Samples were stored in a dark, vacuum environment (˜10 mTorr). Atmospheric exposure was limited to 5-10 minutes before AFM, TEM/STEM, and EELS, respectively. To eliminate liquid traces and minimize hydrocarbon contamination, samples were annealed at 250° C. in 10% H₂/90% Ar for 1 hour prior to TEM/STEM and EELS. All AFM, TEM, STEM, and EELS were performed at room temperature.

Atomic force microscopy (AFM). Suspended few-layer BP flakes were identified on holey SiNₓ membranes using optical microscopy. AFM measurements were carried out in tapping mode using a Bruker Dimension Icon AFM. Thickness measurements of few-layer BP flakes suspended on holey carbon films using the electron cross-section formula can be found in Section S1.

Transmission electron microscopy (TEM). HRTEM images were taken with a JEOL 2010F TEM operating at 200 kV. Nanopores were drilled by fully focusing (i.e. condensing) an electron beam, with probe current and size of 8.0 nA and 1 nm, respectively, for ˜1-2 seconds. Nanoconstrictions and nanogaps were formed by exposing the as-formed nanopores to a broad TEM beam (electron irradiation), corresponding to a current density of around 6.1×10⁻² pA/nm². The instrument's phosphor screen was used to measure the current density. A resolution limit of 0.11 nm is attributed to the microscope (Section S6).

Scanning transmission electron microscopy (STEM). High-angle annular dark-field (HAADF) STEM images were taken with a CEOS aberration-corrected JEOL ARM 200CF TEM. Unless otherwise noted, all HAADF STEM images were taken at 200 kV. Nanopores were first drilled in the spot mode for ˜1-2 minutes with a probe current and size of ˜300 pA and 0.15 nm, respectively. Pores were controllably sculpted into BPNRs and subsequently nanogaps by directing the beam along the nanopore edges in spot mode using a 0.1 nm probe with 14 pA current. This probe was also used during STEM imaging (rastering). Intensity cross-sections of HAADF STEM BPNR images were fitted with moving average profiles and a 0.10 sampling proportion. BPNR thicknesses determined with these cross-sections include an error of ±0.2 nm. Samples were tilted to the [001] zone axis prior to sculpting and imaging. A resolution limit of 0.08 nm is attributed to the microscope (Section S6).

Selected area electron diffraction (SAED). SAED images were taken in a JEOL 2010F operating at 200 kV. A selected area aperture of effective diameter 1 μm was used. SAED patterns were used to tilt samples to the [001] zone axis prior to sculpting and imaging.

Electron energy-loss spectroscopy (EELS). EELS spectra were collected using a Gatan Imaging Filter EELS spectrometer attached to a JEOL 2010F TEM operating at 200 kV. Core-less spectra were recorded with a 1.0 nm STEM probe (current=0.5 nA), energy dispersions of 0.2 eV/channel, and a collection time of 1.0 second.

Theoretical Methods. The Vienna Ab initio Simulation Package (VASP) was used for plane-wave DFT calculations. The generalized gradient approximation (GGA) was implemented via the Perdew-Burke-Ernzerhof (PBE) exchange correlation functional in conjunction with the optB86b functional to include the van der Waals interaction.

An energy cutoff of 500 eV was used for the projector augmented wave (PAW) pseudopotentials. For bulk black phosphorus the atoms and cell were relaxed to critical forces of 0.001 eV/Å using a Monkhorst-Pack k-point sampling of 9×12×4, which resulted in the optimized lattice constants of a=4.35 Å (armchair), b=3.33 Å (zigzag), and c=10.51 Å. To form monolayer phosphorene, four atoms were removed from the bulk unit cell and a vacuum layer of 20 Å was used to prevent interaction between periodic images. The atoms were relaxed to cutoff forces of 0.001 eV/Å using a k-point sampling of 9×12×1.

Nanoribbons were formed by using the lattice vectors and atomic coordinates from the phosphorene sheet. For armchair and both zigzag terminations the unit cell contained one primitive unit cell parallel to the edge, and for both 2×1 supercell reconstructions of the ZZ-2 edge the unit cell contained two primitive unit cells parallel to the edge. For all cases the unit cell contained eight primitive unit cells perpendicular to the edge. An in-plane vacuum of about 20 Å was formed to minimize interaction between periodic images of the nanoribbons. These nanoribbon coordinates were relaxed to a force cutoff of 0.01 eV/Å using k-point samplings of 1×9×1 for armchair, 1×12×1 for both zigzag terminations, and 1×6×1 for both ZZ-2 reconstructions.

The energy barriers for removing single atoms from the phosphorene edges were computer by using the nudged elastic band (NEB) method in the VTST version of VASP with an improved tangent estimate. Sixteen intermediate steps (images) were formed using linear interpolation, with smaller shifts in the low separation region where any potential energy barriers were expected to form. The already existing ribbons were extended perpendicular to the atom removal direction by three unit cells for armchair and both terminations of zigzag, and by two unit cells for the ZZRC edges. This ensures negligible interaction between periodic images of the atom being removed. The removed atom is one which is closest to the edge (see FIG. 2a or FIG. 12) and is shifted up to about 10 Å from the edge to ensure converged energies. The endpoint geometries were converged to a force cutoff of 0.01 eV/Å with k-point samplings of 1 ×3×1 for all cases. Atoms on the edge opposite where the atom was removed were held fixed to speed up convergence and the NEB algorithm was converged to a force cutoff of 0.05 eV/Å.

To form the nanoribbons used in calculating band structures the geometries were converged to force cutoffs of 0.01 eV/Å and the number of k-points was chosen such that the product of the lattice vector and number of k-points was at least as large as the same value for the bulk. An approximate 10 Å in-plane vacuum was used to prevent interaction between periodic images. At least five bands were used per atom to ensure well-converged bands and 200 k-points were used along the high symmetry directions, found using the AFLOW tool.

To calculate the charge densities for the edge, bands structures were re-computed with 25 k-points along the high symmetry directions. Since the Kohn-Sham energy eigenvalues are output in ascending order for each k-point, the band numbering changes if bands cross. To circumvent this, regions in the band structures were identified where only the suspected edge bands crossed over each other and the corresponding k-points in these regions were included in the partial charge density calculations.

Thickness Analysis of Few-Layer BP Flakes on Holey Carbon Grids (S1).

To estimate the thickness of few-layer BP flakes suspended on holey carbon TEM grids, the simplified electron cross-section formula for HAADF STEM imaging was used:

$$\frac{{I}_{p} - {I}_{b}}{{I}_{c} - {I}_{b}} = {( \frac{{Z}_{p}}{{Z}_{c}} )}^{2} \frac{{ρ}_{p} {t}_{p}}{{ρ}_{c} {t}_{c}}$$

Where Iₚ, I_{c}, and I_{b} are the mean image intensities of the few-layer BP flake, carbon film, and background, respectively, as shown in FIG. 6. ρₚ(2.69 g/cm³) and ρ_{c}(2.15 g/cm³) are the densities of black phosphorus and amorphous carbon, respectively, while Zₚ(15) and Z_{c}(6) are the corresponding atomic numbers. t_{c} is the known thickness (20 nm) of the carbon support film and tₚ is the unknown thickness of the flake, respectively. The resulting thickness of 17 nm for the flake in FIG. 6 is in good agreement with the sub-20 nm flake thicknesses obtained on holey SiNₓ membranes via AFM.

BP STEM Image Simulations (S2).

STEM images were simulated using the QSTEM package. The relaxed geometries of phosphorene and bulk BP obtained from the DFT calculations were used to construct the atomic models. Potential slices were chosen so that single planes of atoms were included in each: two slices for phosphorene and four slices for the unit cell of bulk BP. An array of 200×200 pixels was used and the scattering angle (determined by the probe array size) was set greater than the largest detector angle. The aberration-corrected STEM device parameters used in the simulation were: Acceleration Voltage: 200 kV; Defocus: 1.729 nm; Astigmatism: 1.613 nm, −60.5 degrees; Spherical Aberration C3: −580 nm; Chromatic Aberration: 1.1 mm; Aberration Spread: 0.4 eV;

Convergence Angle: 25 mrad; Detector Inner Angle: 50 mrad; Detector Outer Angle: 180 mrad.

The resulting STEM images for phosphorene and AB stacked bilayer BP (single unit cell of bulk BP) are shown in FIGS. 7a and FIG. 7b, respectively. The observation of a “half lattice constant” scheme indicates the samples are at least bilayer.

Width dependence of thermodynamic stability for 2D and bulk armchair, ZZ-1, and ZZ-2 edge nanoribbons from the band structure calculations (S10)

For a 2D system the thermodynamic stability is quantified by the edge energy, which is calculated as (F₀-NFₚ)/2L, where F₀ is the total free energy of the system, N is the number of phosphorus atoms, Fp is the free energy per atom for phosphorene, and L is the lattice constant in the direction along the edge. Similarly, for the bulk system one may calculate the surface energy as (F₀-NF_{BP})/2A, where F_{BP} is the free energy per atom for bulk BP, A is the unit cell area on the surface, and the other symbols are defined similarly. FIG. 19 provides thermodynamic stability of 2D and bulk armchair, ZZ-1, and ZZ-2 nanoribbons as a function of width.

### Illustrative Embodiments

In one aspect, the present disclosure provides analysis devices. A device may comprise a portion of black phosphorous having a region that comprises one or more (e.g., from 1 to about 100) layers of black phosphorous; the region having at least one pore formed therehrough,and the at least one pore having a cross-sectional dimension in the range of from about 1 to about 100 nm.

A pore may have a cross-sectional dimension in the range of from about 1 to about 50 nm, or from about 1 to about 20 nm, or even from about 1 to about 10 nm. A pore may be cylindrical or conical, but may also be polygonal or even hourglass-shaped.

The portion of black phosphorous may supported at least one location by a support membrane. A support membrane may include one or more apertures, and the pore of the region of black phosphorous may be in register with an aperture of the support membrane. A support membrane may be “holey” in nature. The apertures of the support membrane may be regular/periodic in nature, but may also be non-periodic in their positioning.

Suitable support membrane materials include, e.g., Si, SiNx, C, or any combination thereof. SiNx membranes are considered particularly suitable.

The region of black phosphorous may comprise from, e.g., 1 to about 50 layers of black phosphorous, e.g., 1, 2, 3, 4, 5, 6, 7, 8, 9, 10, 11, 12, 13, 14, 15, 16, 17, 18, 19, or even 20 layers. Regions that comprise one or two (or event three) layers are considered especially suitable, but are not required.

The region of black phosphorous may comprise a region of armchair structure, a region of zig-zag structure, or any combination thereof The armchair and/or zig zag structure may be present at any location within the region of black phosphorous. In some embodiments, at least a portion of an edge of the region of black phosphorous is characterized as having an armchair structure. In some embodiments, at least a portion of an edge of the region of black phosphorous is characterized as having an zig-zag structure.

An armchair portion may be present within the bulk of the region, i.e., spanning all or some of a distance between edges of the region. Likewise, a zig zag portion may be present within the bulk of the region, i.e., spanning all or some of a distance between edges of the region. Both armchair and zig zag regions may be present.

In some embodiments, at least a portion of an edge of the region of black phosphorous is characterized as being non-linear in structure. An edge (or a portion thereof) may be characterized as being jagged in structure. An edge may be saw-toothed, polygonal, or otherwise non-linear in structure.

The region of black phosphorous may have a cross-sectional dimension in the range of from about 1 to about 10,000 nm, e.g., from about 50 to about 1000 nm, or from about 100 to about 500 nm. It should be understood that width, height, radius, and diameter are all considered cross-sectional dimensions. The region may include a narrowed region that includes a portion that is less wide than other portions of the region, e.g., a dog-bone shape. In some embodiments, the region may be rectangular in shape. A region may also be polygonal, circular or oblong in structure. As one example, the amount of black phosphorous may include a narrowed region (e.g., a ribbon) in which one or more pores reside.

The present disclosure also provides methods of molecular analysis. These methods may comprise contacting an analysis device according to the present disclosure to a sample comprising one or more molecules; translocating one or more of the molecules through the pore of the analysis device; and monitoring a signal related to the translocation of the molecule through the pore.

Some exemplary molecules are, e.g., DNA (single- and double-stranded), RNA (single- and double-stranded), proteins, polymers (biological and synthetic), and the like.

The methods may also comprise correlating the signal to one or more structural features of the molecule. A structural feature may be, e.g., size, length, nucleotide composition, or other structural feature (including chemical composition).

A signal may be an electrical signal. Suitable electrical signals are, e.g., one or more of a current, a voltage, a resistance, or any combination thereof.

The translocation is effected by applying a gradient to the molecule. Chemical, mechanical (e.g., pressure), and electrical gradients are all considered suitable.

Additionally provided are methods of fabricating an analysis device. The methods comprise disposing an amount of black phosphorous on a support membrane, the amount of black phosphorous having 1 or more (e.g., from 1 to about 100) layers; and forming a pore in a region of the black phosphorous.

The methods may include exfoliating at least some of the layers of the portion of the amount of black phosphorous. One or more layers may be exfoliated, and the exfoliation may be accomplished by mechanical, chemical, or other means. Mechanical exfoliation is considered especially suitable.

Pore formation may be accomplished by electron beam, mechanical methods, chemical methods, or any combination thereof.

The disclosed methods may also include removing an amount of the black phosphorous so as to reduce a cross-sectional dimension of the amount of black phosphorous. The removing may be performed so as to reduce the cross-sectional dimension to from about 1 to about 10,000 nm, e.g., from about 10 to about 1000 nm, or even from about 50 to about 500 nm. Removal may be effected by electron beam, mechanical methods, chemical methods, or any combination thereof.

The methods may further comprise placing the amount of black phosphorous into electronic communication with an electronic metering device. The electronic metering device is configured to detect one or more of a voltage, a current, a resistance, or any combination thereof.

The methods may be performed so as to give rise to a device according to the present disclosure.

Further provided are methods of molecular analysis, comprising: contacting an analysis device comprising a pore formed in a region of black phosphorous to a sample comprising one or more molecules; translocating one or more of the molecules through the pore of the analysis device; and monitoring a signal related to the translocation of the molecule through the pore.

As described elsewhere herein, a pore may have a cross-sectional dimension in the range of from about 1 to about 100 nm, e.g., from about 1 to about 20 nm. The pore may be formed in a region of black phosphorous that comprises from 1 to 20 layers, from 1 to 5 layers, or even 1 or 2 layers.

Suitable signals are described elsewhere herein. The methods may also include correlating the signal to one or more structural features of the molecule.

### Example Embodiments

Embodiment 1. An analysis device, comprising: a portion of black phosphorous having a region that comprises at least one layer of black phosphorous; the region having at least one pore formed therehrough, the at least one pore having a cross-sectional dimension in the range of from about 1 to about 100 nm.

Embodiment 2. The analysis device of embodiment 1, wherein the portion of black phosphorous is supported at least one location by a support membrane.

Embodiment 3. The analysis device of embodiment 2, wherein the support membrane comprises one or more apertures.

Embodiment 4. The analysis device of embodiment 3, wherein the pore of the region of black phosphorous is in register with an aperture of the support membrane.

Embodiment 5. The analysis device of any of embodiments 1-4, wherein the support membrane comprises Si, SiNx, C, or any combination thereof

Embodiment 6. The analysis device of any of embodiments 1-5, wherein the region of black phosphorous comprises from 1 to about 100 layers of black phosphorous. Regions having from 1-10 layers of black phosphorous are considered suitable.

Embodiment 7. The analysis device of any of embodiments 1-6, wherein the region of black phosphorous comprises a region of armchair structure, a region of zig-zag structure, or any combination thereof.

Embodiment 8. The analysis device of embodiment 7, wherein at least a portion of an edge of the region of black phosphorous is characterized as having an armchair structure.

Embodiment 9. The analysis device of embodiment 7, wherein at least a portion of an edge of the region of black phosphorous is characterized as having an zig-zag structure.

Embodiment 10. The analysis device of any of embodiments 1-9, wherein at least a portion of an edge of the region of black phosphorous is characterized as being non-linear in structure.

Embodiment 11. The analysis device of any of embodiments 1-9, wherein at least a portion of an edge of the region of black phosphorous is characterized as being jagged in structure.

Embodiment 12. The analysis device of any of embodiments 1-11, wherein the region has a cross-sectional dimension in the range of from about 1 to about 10,000 nm.

Embodiment 13. The analysis device of embodiment 12, wherein the region has a cross-sectional dimension in the range of from about 50 to about 1000 nm.

Embodiment 14. The analysis device of embodiment 13, wherein the region has a cross-sectional dimension in the range of from about 100 to about 500 nm.

Embodiment 15. A method of molecular analysis, comprising: contacting an analysis device according to any of embodiments 1-14 to a sample comprising one or more molecules; translocating one or more of the molecules through the pore of the analysis device; and monitoring a signal related to the translocation of the molecule through the pore.

Embodiment 16. The method of embodiment 15, further comprising correlating the signal to one or more structural features of the molecule.

Embodiment 17. The method of any of embodiments 15-16, wherein the signal is an electrical signal.

Embodiment 18. The method of embodiment 17, wherein the electrical signal is one or more of a current, a voltage, a resistance, or any combination thereof.

Embodiment 19. The method of any of embodiments 15-18, wherein the translocation is effected by applying a gradient to the molecule.

Embodiment 20. The method of embodiment 20, wherein the gradient is an electrical gradient.

Embodiment 21. A method of fabricating an analysis device, comprising: disposing an amount of black phosphorous on a support membrane, the amount of black phosphorous having at least one layer; and forming a pore in a region of the black phosphorous.

Embodiment 22. The method of embodiment 21, further comprising exfoliating at least some of the layers of the portion of the amount of black phosphorous. The foregoing may be applied to embodiments where the amount of black phosphorous is a single layer, but may also be applied to embodiments where the amount of black phosphorous comprises a plurality of layers.

Embodiment 23. The method of any of embodiments 21-22, further comprising removing an amount of the black phosphorous so as to reduce a cross-sectional dimension of the amount of black phosphorous.

Embodiment 24. The method of embodiment 23, wherein the removing is performed so as to reduce the cross-sectional dimension to from about 1 to about 10,000 nm.

Embodiment 25. The method of any of embodiments 21-24, further comprising placing the amount of black phosphorous into electronic communication with an electronic metering device.

Embodiment 26. The method of embodiment 25, wherein the electronic metering device is configured to detect one or more of a voltage, a current, a resistance, or any combination thereof.

Embodiment 27. The method of any of embodiments 21-26, wherein the method is performed so as to give rise to a device according to any of embodiments 1-14.

Embodiment 28. A method of molecular analysis, comprising: contacting an analysis device comprising a pore formed through a region of black phosphorous to a sample comprising one or more molecules; translocating one or more of the molecules through the pore of the analysis device; and monitoring a signal related to the translocation of the molecule through the pore.

Embodiment 29. The method of embodiment 28, wherein the region of black phosphorous is supported by a support membrane.

Embodiment 30. The method of any of embodiments 28-29, wherein the pore extends through from 1 to about 5 layers of black phosphorous.

The foregoing disclosure is illustrative only and does not limit the scope of the following claims.

1. An analysis device, comprising:
a portion of black phosphorous having a region that comprises at least one layer of black phosphorous;
the region having at least one pore formed therehrough, the at least one pore having a cross-sectional dimension in the range of from about 1 to about 100 nm.

2. The analysis device of claim 1, wherein the portion of black phosphorous is supported at least one location by a support membrane.

3. The analysis device of claim 2, wherein the support membrane comprises one or more apertures.

4. The analysis device of claim 3, wherein the pore of the region of black phosphorous is in register with an aperture of the support membrane.

5. The analysis device of claim 1, wherein the support membrane comprises Si, SiNx, C, or any combination thereof.

6. The analysis device of claim 1, wherein the region of black phosphorous comprises from 1 to about 100 layers of black phosphorous.

7. The analysis device of any of claim 1, wherein the region of black phosphorous comprises a region of armchair structure, a region of zig-zag structure, or any combination thereof.

8. The analysis device of claim 7, wherein at least a portion of an edge of the region of black phosphorous is characterized as having an armchair structure.

9. The analysis device of claim 7, wherein at least a portion of an edge of the region of black phosphorous is characterized as having an zig-zag structure.

10. The analysis device of claim 1, wherein at least a portion of an edge of the region of black phosphorous is characterized as being non-linear in structure.

11. The analysis device of claim 1, wherein at least a portion of an edge of the region of black phosphorous is characterized as being jagged in structure.

12. The analysis device of claim 1, wherein the region has a cross-sectional dimension in the range of from about 1 to about 10,000 nm.

13. (canceled)

14. (canceled)

15. A method of molecular analysis, comprising:
contacting an analysis device to a sample comprising one or more molecules,
the analysis device comprising a portion of black phosphorous having a region that comprises at least one layer of black phosphorous, the region having at least one pore formed therehrough, and the at least one pore having a cross-sectional dimension in the range of from about 1 to about 100 nm;
translocating one or more of the molecules through the pore of the analysis device; and
monitoring a signal related to the translocation of the molecule through the pore.

16. The method of claim 15, further comprising correlating the signal to one or more structural features of the molecule.

17. The method of any of claim 15, wherein the signal is an electrical signal.

18. The method of claim 17, wherein the electrical signal is one or more of a current, a voltage, a resistance, or any combination thereof.

19. The method of claim 15,wherein the translocation is effected by applying a gradient to the molecule.

20. (canceled)

21. (canceled)

22. (canceled)

23. (canceled)

24. (canceled)

25. (canceled)

26. (canceled)

27. (canceled)

28. A method of molecular analysis, comprising:
contacting an analysis device comprising a pore formed through a region of black phosphorous to a sample comprising one or more molecules;
translocating one or more of the molecules through the pore of the analysis device; and
monitoring a signal related to the translocation of the molecule through the pore.

29. The method of claim 28, wherein the region of black phosphorous is supported by a support membrane.

30. The method of any of claim 28, wherein the pore extends through from 1 to about 5 layers of black phosphorous.
